(19) United States
(12) Patent Application Publication
(10) Pub. No.: US 2004/0213831 A1
(43) Pub. Date: Oct. 28, 2004

(54) Tts with peel-off film with reduced resistance to tearing

(76) Inventor: Ralf Kibele, Holzkirchen (DE)

Correspondence Address: Marshall Gerstein & Borun, 6300 Sears Tower, 233 South Wacker Drive, Chicago, IL, 60606-6357, US

(21) Appl. No.: 10/480,015

(22) PCT Filed: Jun. 6, 2002
(86) PCT No.: PCT/EP02/06215

(30) Foreign Application Priority Data
Jun. 7, 2001 (DE) 10127455.6

(51) Int. Cl. A61F 13/02

(52) U.S. Cl. 424/448; 604/180

(57) ABSTRACT

The invention relates to a transdermal therapeutic system (TTS) which is provided with a peel-off film which exhibits a reduced resistance to tearing in a (1 dimensional direction).

Peel-off aids or application aids are widely used in transdermal therapeutic systems that are on the market. Reference is made by way of example to PCT/EP 97/06 799. Peel-off aids are intended to enable the user to apply the transdermal therapeutic system more easily. Peel-off aids are, therefore, at least a useful element of the system, since use of the system is facilitated by peel-off aids.

In the case of delicate transdermal therapeutic systems, however, direct formation of the peel-off aid may a) damage the system or b) adversely affect the stability of the system.

Case a) occurs especially with transdermal therapeutic systems of the reservoir type, in which, as a result of damage to the membrane, the solution containing the active ingredient can escape from the reservoir, thereby rendering the system useless. Case b) occurs especially with transdermal therapeutic systems of the matrix type, in which, because of the nature of the system, the system is usually over-saturated with the relevant active ingredient. It has been possible to observe, for example, that crystallisation of the active ingredient occurs to a greater extent along the cut or the punched area of the peel-off aid.

If a peel-off aid is not provided in the case of transdermal therapeutic systems of the reservoir type, use is made considerably more difficult.

For case b), frequently a laminate with a first peel-off film is made first of all which in the course of manufacture is peeled off and replaced by a second peel-off film having a pre-punched peel-off aid. The production of a transdermal therapeutic system of the matrix type with a first and a second peel-off film leads, however, to increased costs, increased expenditure in terms of machinery and increased production times.

In order to improve the prior art, there is provided according to the invention a transdermal therapeutic system (TTS) having a peel-off film that has a reduced resistance to tearing or tear propagation in one direction (1-dimensionally).

Suitable peel-off films have, for example, a tear propagation resistance (1-dimensionally) of from 10 to 50, and especially from 15 to 25, newtons/mm.

According to the invention, the peel-off film may project all round beyond the periphery of the transdermal therapeutic system or the remainder of the construction elements of the transdermal therapeutic system. The expression “remainder of the construction elements” is understood in the present context to mean all the construction elements of a transdermal therapeutic system apart from the peel-off film.

According to the invention, the all-round projection of the peel-off film may have a peel-off aid for tearing the peel-off film in the direction of the reduced resistance to tearing.

According to the invention, the peel-off aid may be an incision, a notch, a perforation or a weak site. That peel-off aid may have been provided by punching.

According to the invention, the peel-off aid may be provided starting at the edge of the peel-off film.

According to the invention, the peel-off aid may extend up to the transdermal therapeutic system (the remainder of its construction elements).

According to the invention, the peel-off film may have, in addition to a first peel-off aid, a further peel-off aid which is provided on the opposite side of the transdermal therapeutic system (the remainder of its construction elements).

According to the invention, the two peel-off aids may be so arranged relative to each other that the end of a tear that begins at one of the two peel-off aids divides the other peel-off aid upon peeling-off of the peel-off film.

According to the invention, the peel-off film may be a biaxially oriented or stretched film or a film composite comprising a biaxially oriented or stretched film.

According to the invention, the single-layer peel-off film or at least one film of the film composite may be a polypropylene or polyethylene film (PP or PE film), especially a blown poly-propylene film. Blown polypropylene films (PPb films) are superior to cast PP films (PPc films) or polyethylene films as a basic material since blown polypropylene films have a higher temperature stability (less shrinkage) and usually a more uniform layer thickness.

Biaxially stretched polypropylene films have a high resistance to transverse tearing but virtually no resistance to longitudinal tear propagation. According to the invention, that property can advantageously be exploited for peel-off aids.

According to the invention, the film composite may be a PP/PE, PP/PET, PP/PS, PP/EVA, PP/EVOH, PP/paper, PP/PVC, PP/PVDC or PP/OPA composite.

For a PP/PET composite a PET thickness of not more than about 15 μm is to be preferred.

In the case of a film composite, the main component in terms of thickness is preferably PP. According to the invention, the polypropylene component of the film composite is as thick or thicker than the other components of the film composite.

According to the invention, a transdermal therapeutic system may be provided with a peel-off film having a thickness of from 15 to 300 μm and especially from 50 to 150 μm.

According to the invention, a transdermal therapeutic system may be provided in which the peel-off film in the form of a single-layer film or in the form of a film composite is provided with a separation coating. The separation coating may be a silicone treatment or a fluorosilicone treatment.

According to the invention, therefore, a simple straight cut provided at any desired position at the edge of the projection of the peel-off film, longitudinally with respect to the running direction, or rather in the direction of reduced tear propagation resistance, advantageously makes it possible for a peel-off aid to be provided without the transdermal therapeutic system being damaged. By a simple tug, the peel-off film can be separated into two parts in a defined manner upon application.

The invention is described in detail below with reference to Figures and Examples.

In the Figures:

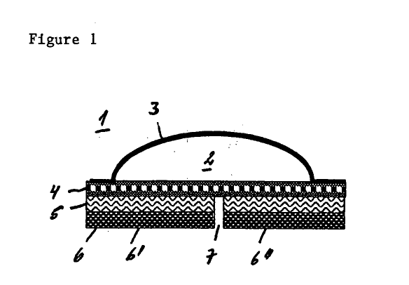

FIG. 1 shows a prior art TTS of the reservoir type;

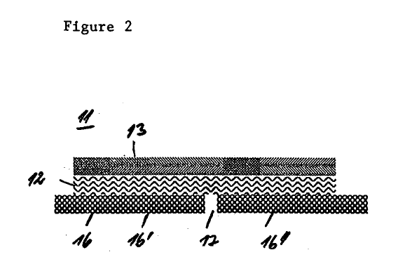

FIG. 2 shows a prior art TTS of the matrix type;

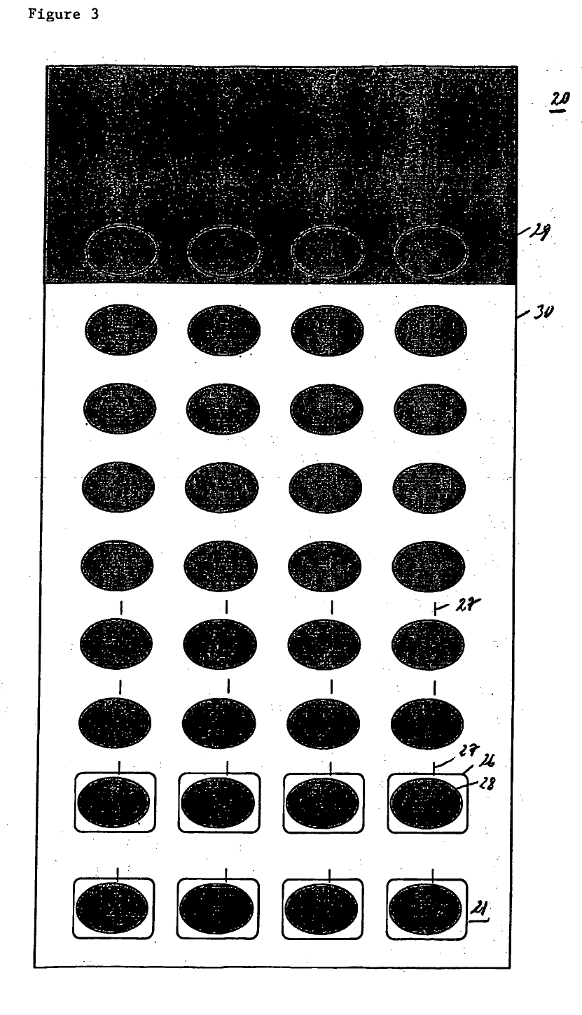

FIG. 3 shows various stages in the production of transdermal therapeutic systems according to the invention;

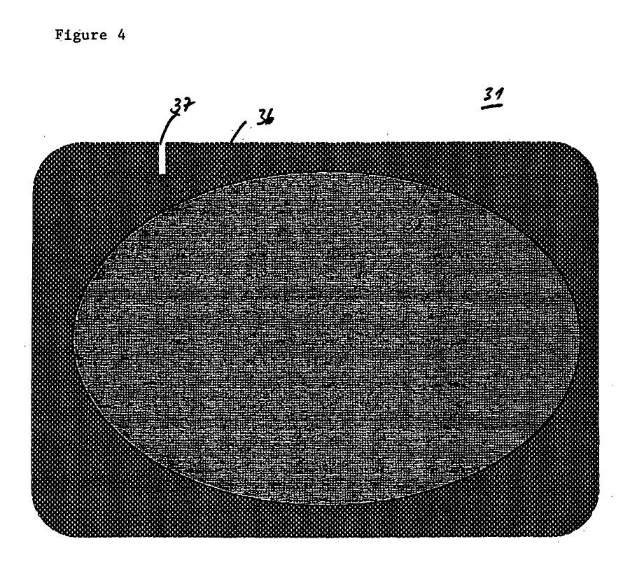

FIG. 4 shows a TTS according to the invention with one peel-off aid;

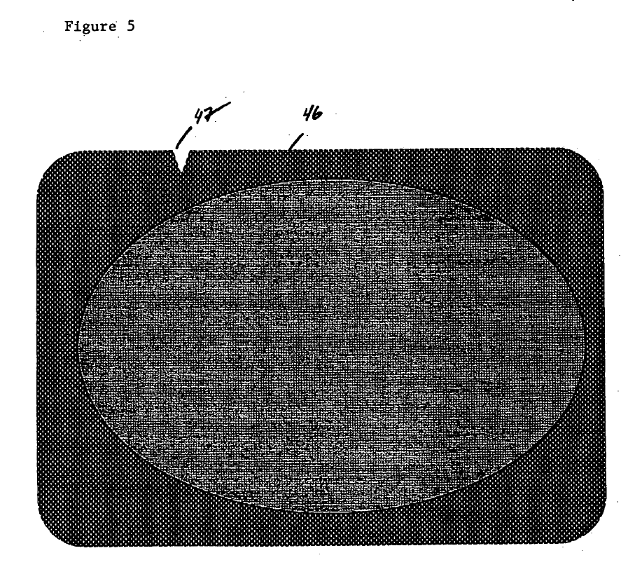

FIG. 5 shows a TTS according to the invention with one peel-off aid;

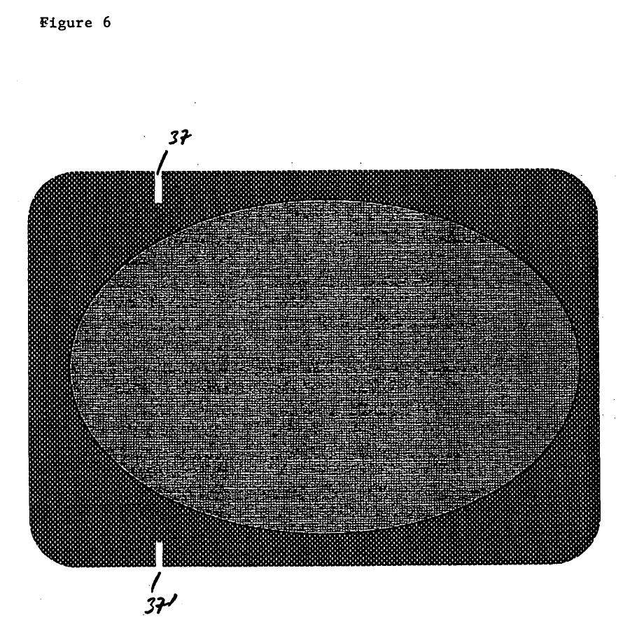

FIG. 6 shows a TTS according to the invention with two peel-off aids;

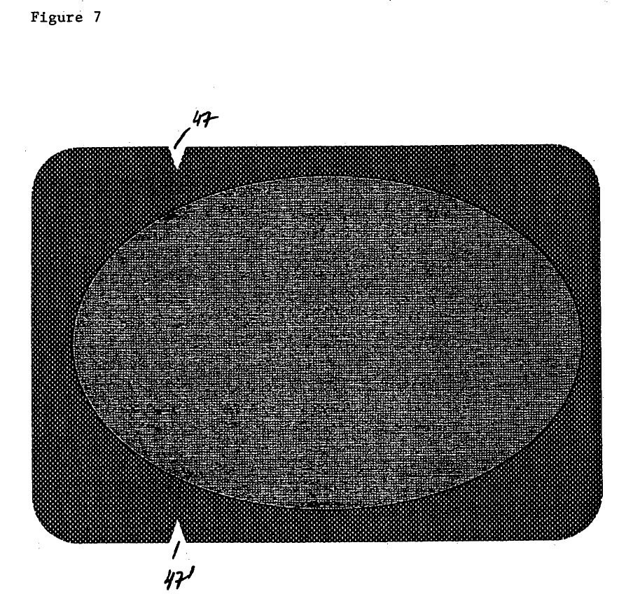

FIG. 7 shows a TTS according to the invention with two peel-off aids; and

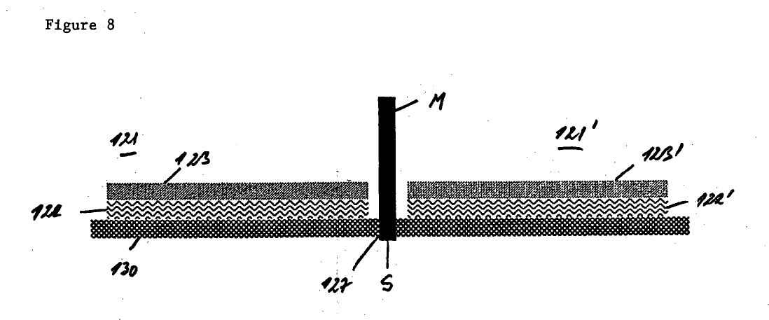

FIG. 8 shows a stage in the production of therapeutic systems according to the invention each having one peel-off aid.

In the case of the transdermal therapeutic system 1 shown in FIG. 1, the reservoir 2, which contains the active ingredient to be administered, is enclosed by a covering film 3 and a membrane 4. A self-adhesive layer 5, which is protected by a peel-off film 6 until application of the TTS 1, is applied to the planar membrane 4. The peel-off film 6 has a cut 7 which runs across the peel-off film. 6 and which may extend into the self-adhesive layer 5. That cut 7 may be formed, for example, in accordance with PCT/EP 97/06799. The cut 7 allows the TTS 1 to be flexed slightly during application in such a way that the peel-off film 6 protrudes slightly in a convex shape and the cut edges of the cut 7 project in such a manner that they can be gripped and the two parts of the peel-off film 6′, 6″ can be peeled off the adhesive layer 5 so that the adhesive layer 5 is exposed and the TTS 1 can be applied.

In the case of the prior art TTS 11 of the matrix type shown in FIG. 2, a matrix layer 12 containing the active ingredient is applied to a covering film 13 and is covered on the side thereof remote from the covering film 13 with a peel-off film 16. The covering film 16 is again provided with a continuous incision 17 which allows the peel-off film 16 to be peeled off in halves. The two halves 16′, 16″ of the peel-off film 16 correspond to the halves 6′, 6″ of the peel-off film 6 of the TTS 1 shown in FIG. 1.

FIG. 3 is intended to illustrate the production of a TTS according to the invention of the matrix type. The web 20 may be provided in the form of a three-layered continuous band (region 29), the top layer of the web 20 being provided for the covering films, the middle layer of the web 20 for the matrix layers and the bottom layer of the web 20 for the peel-off films of the transdermal systems that are to be produced. The three-layered web 20 is provided with approximately elliptical incisions (arranged in four rows) which extend through the top and middle layers of the web 20 and define individual patches 28. Thereafter, the top and middle layers of the web 20 are peeled off in such a manner that only the bottom layer 30 with the pre-formed patches 28 (arranged in four rows) remains. In a further step, the bottom layer 30 of the web 20 is provided with incisions 27, more specifically with one incision 27 per patch 28 to be produced. In a further step, approximately rectangular peel-off films 26 are punched out of the bottom web 30 in such a manner that the individual peel-off films 26 project all round beyond the periphery of the patches 28 formed by covering film and matrix (patches without peel-off film 26). It can be seen from FIG. 3 that the incisions 27 are provided in such a manner that each extends towards its associated patch 28 and through the edge of the peel-off film 26 but does not reach the periphery of its respectively associated patch 28.

This situation can be seen in detail from FIG. 4. FIG. 4 shows a transdermal system 31 of the matrix type which is approximately elliptical in plan view and which is formed by a covering film and a self-adhesive matrix layer lying beneath the latter, the matrix layer being covered by a peel-off film 36 which projects all round. That peel-off film 36 is provided with an incision 37 that cuts into the peel-off film 36 from the edge of the peel-off film 36 but does not reach the patch 38 (patch without peel-off film). According to the invention, the peel-off film 36 has a reduced resistance to tearing in one direction (1-dimensionally). Since the incision 37 extends in the direction of the reduced resistance to tearing, upon application of the transdermal system 31 the peel-off film 36 can be divided beneath the patch 37 by means of the incision 37 in such a manner that the two parts of the peel-off film 36 which, upon tearing of the incision 37, remain behind on both sides of the tear can easily be removed from the patch 38.

FIG. 5 shows a TTS according to the invention in which the peel-off aid 48 has been formed as a wedge-shaped incision in the peel-off film 46.

FIGS. 6 and 7 show transdermal therapeutic systems each of which has two peel-off aids (37, 37′ in FIG. 6 and 47, 47′ in FIG. 7) each provided at the edge of the peel-off film so that they do not reach the patch formed by covering film and matrix. The peel-off aids 37, 37′ and 47, 47′ are thus arranged in pairs and in such a manner that a tear beginning at one of the peel-off aids and taken in the direction of the reduced resistance to tearing of the peel-off film will in each case divide the opposite peel-off aid.

FIG. 8 shows two transdermal therapeutic systems 121, 121′ each of which is formed in two layers by a covering film 123, 123′ and a self-adhesive matrix layer 122, 122′. The two transdermal systems 121, 121′ are provided on a common web 130 from which peel-off films, which carry the patches 123, 123′ and project all round, are punched according to FIG. 3. FIG. 8 shows a step in the TTS production, which step precedes the punching out of the peel-off films according to FIG. 3 and in which an incision 127 is made by a tool M having a cutting edge S lying in the plane of the drawing, which incision 127 extends merely through the web 130 and not, however, through the patches 123, 123′ and leaves those patches 123, 123′ intact. In the illustrative embodiment shown, a peel-off aid for the patch 123 and a peel-off aid for the patch 123′ are made simultaneously by a single incision 127 before the peel-off films for the two patches 123, 123′ are punched out of the still common web 130.

1. A transdermal therapeutic system having a peel-off film that has a reduced resistance to tearing in one direction.

2. A transdermal therapeutic system according to claim 1, wherein the peel-off film projects all round beyond the periphery of the remainder of the construction elements of the transdermal therapeutic system.

3. A transdermal therapeutic system according to claim 1, wherein the all-round projection of the peel-off film has a peel-off aid for tearing the peel-off film in the direction of the reduced resistance to tearing.

4. A transdermal therapeutic system according to claim 3, wherein the peel-off aid is an incision, a notch, a perforations or a weak site.

5. A transdermal therapeutic system according to claim 3, wherein the peel-off aid is provided starting at an edge of the peel-off film.

6. A transdermal therapeutic system according to claim 3, wherein the peel-off aid extends up to the remainder of the construction elements of the transdermal therapeutic system.

7. A transdermal therapeutic system according to claim 3, wherein the all-round projection of the peel-off film has, in addition to a first peel-off aid, a second peel-off aid which is provided on an opposite side of the remainder of the construction elements of the transdermal therapeutic system from the first peel-off aid.

8. A transdermal therapeutic system according to claim 7, wherein the first and second peel-off aids are so arranged relative to each other that an end of a tear that begins at one of the two peel-off aids divides the other peel-off aid upon peeling-off the peel-off film.

9. A transdermal therapeutic system according to claim 1, wherein the peel-off film is a biaxially stretched film or a film composite comprising a biaxially stretched film.

10. A transdermal therapeutic system according to claim 9, wherein the single-layer peel-off film or at least one layer of the film composite is a polypropylene (PP) or polyethylene (PE) film.

11. A transdermal therapeutic system according to claim 1, wherein the film composite is a composite selected from the group consisting of polypropylene/polyethylene, polypropylene/polyethylene terephthalate, polypropylene/polystyrene, polypropylene/ethylene, vinyl acetate, polypropylene/ethylene vinyl alcohol , polypropylene/paper, polypropylene/polyvinyl chloride, polypropylene/polyvinyl dichloride, and polypropylene/oriented polyamide composites.

12. A transdermal therapeutic system according to claim 11, wherein the polypropylene component of the film composite is at least as thick as the other component of the film composite.

13. A transdermal therapeutic system according to claim 1, comprising a peel-off film having a thickness of from 15 μm to 300 μm.

14. A transdermal therapeutic system according to claim 1, wherein the peel-off film is provided with a separation coating.

15. A transdermal therapeutic system according to claim 14, wherein the separation coating is a silicone treatment or a fluorosilicone treatment.

16. A transdermal therapeutic system according to claim 10, wherein the single layer peel-off film is at least one layer of the film composite is a blown polypropylene film.

17. A transdermal therapeutic system according to claim 13, comprising a peel-off film having a thickness of from 50 μm to 150 μm.
